(19) United States
(12) Patent Application Publication
(10) Pub. No.: US 2020/0035932 A1
(43) Pub. Date: Jan. 30, 2020

(54) PHOTOELECTRIC CONVERSION ELEMENT, OPTICAL SENSOR, AND IMAGING ELEMENT

(71) Applicant: FUJIFILM Corporation, Tokyo (JP)

(72) Inventors: Tomoaki YOSHIOKA, Kanagawa (JP); Eiji FUKUZAKI, Kanagawa (JP); Kimiatsu NOMURA, Kanagawa (JP)

(73) Assignee: FUJIFILM Corporation, Tokyo (JP)

(21) Appl. No.: 16/592,785

(22) Filed: Oct. 4, 2019

Related U.S. Application Data
(63) Continuation of application No. PCT/JP2018/014250, filed on Apr. 3, 2018.

(30) Foreign Application Priority Data
Apr. 7, 2017 (JP) 2017-076540
Jan. 25, 2018 (JP) 2018-010365

(51) Int. Cl. H01L 51/00; C07D 487/22

(52) U.S. Cl. CPC H01L 51/0092; C07D 487/22; H01L 51/4253; H01L 51/008; H01L 51/0046; H01L 51/0068

(57) ABSTRACT

The present invention provides a photoelectric conversion element exhibiting excellent responsiveness, and excellent dark current characteristics in a case of high-speed photoelectric conversion film formation, an optical sensor, an imaging element, and a compound which include the photoelectric conversion element. The photoelectric conversion element of the present invention includes a conductive film, a photoelectric conversion film, and a transparent conductive film, in this order, in which the photoelectric conversion film contains a compound represented by Formula (1), and an n-type organic semiconductor having a predetermined structure.

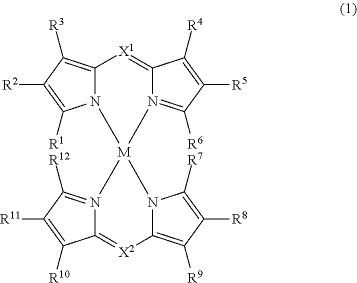

### CROSS-REFERENCE TO RELATED APPLICATIONS

This application is a Continuation of PCT International Application No. PCT/JP2018/014250 filed on Apr. 3, 2018, which claims priority under 35 U.S.C. § 119(a) to Japanese Patent Application No. 2017-076540 filed on Apr. 7, 2017 and Japanese Patent Application No. 2018-010365 filed on Jan. 25, 2018. Each of the above applications is hereby expressly incorporated by reference, in its entirety, into the present application.

### BACKGROUND OF THE INVENTION

### 1. Field of the Invention

The present invention relates to a photoelectric conversion element, an optical sensor, and an imaging element.

### 2. Description of the Related Art

In the related art, a planar solid-state imaging element in which photodiodes (PDs) are two-dimensionally arranged and a signal charge generated in each PD is read out by a circuit is widely used as a solid-state imaging element.

In order to realize a color solid-state imaging element, a structure in which a color filter transmitting light of a specific wavelength is disposed on a light incident surface side of the planar solid-state imaging element is generally used. Currently, a single plate solid-state imaging element in which the color filter transmitting blue (B) light, green (G) light, and red (R) light is regularly arranged on each PD which is two-dimensionally arranged is well known. However, in this single plate solid-state imaging element, light which is not transmitted through the color filter is not used, thus light utilization efficiency is poor.

In order to solve these disadvantages, in recent years, development of a photoelectric conversion element having a structure in which an organic photoelectric conversion film is disposed on a substrate for reading out a signal has progressed. US2014/0097416A discloses, for example, a photoelectric conversion element having a photoelectric conversion film containing the following compounds as such a photoelectric conversion element using the organic photoelectric conversion film.

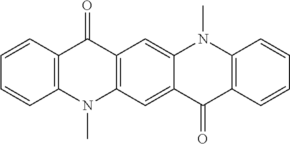 [SMILES: Cn1c2ccccc2c(=O)c2cc3c(cc21)c(=O)c1ccccc1n3C]

### SUMMARY OF THE INVENTION

In recent years, further improvements are also required for various characteristics required for a photoelectric conversion element used in an imaging element and an optical sensor, along with demands for improving performance of the imaging element, the optical sensor, and the like.

For example, further improvement in responsiveness is required.

Further, from the viewpoint of producing suitability, it is required to keep a value of a dark current of the photoelectric conversion element low even in a case where the photoelectric conversion film is formed at high speed.

The inventor of the present invention has produced a photoelectric conversion element using a compound (for example, the above-described compound) specifically disclosed in US2014/0097416A, and has examined about the responsiveness of the obtained photoelectric conversion element, and the value of the dark current of the photoelectric conversion element in a case where the photoelectric conversion film is formed at high speed (hereinafter, also simply referred to as “dark current characteristics in a case of high-speed film formation”). As a result, the inventor has found that the characteristics do not necessarily reach the level required recently and further improvement is necessary.

In the latter stage, the fact that the value of the dark current is small refers to that the dark current characteristic in a case of high-speed film formation is excellent.

In view of the above-described circumstances, an object of the present invention is to provide a photoelectric conversion element exhibiting excellent responsiveness, and excellent dark current characteristics in a case of high-speed film formation.

Another object of the present invention is to provide an optical sensor, an imaging element, and a compound which include the photoelectric conversion element.

The inventor of the present invention has conducted extensive studies on the above-described problems. As a result, the inventor has found that it is possible to solve the above-described problems using a photoelectric conversion film containing a compound having a predetermined structure, and has completed the present invention.

That is, the above-described problems can be solved by means shown below.

(1) A photoelectric conversion element comprising a conductive film, a photoelectric conversion film, and a transparent conductive film, in this order, in which the photoelectric conversion film contains a compound represented by Formula (1) described below, and an n-type organic semiconductor, and the n-type organic semiconductor contains at least one selected from the group consisting of a compound represented by Formula (2) described below and a compound represented by Formula (3) described below.

(2) The photoelectric conversion element according to (1), in which the n-type organic semiconductor contains the compound represented by Formula (3) described below.

(3) The photoelectric conversion element according to (1) or (2), in which M represents Zn, Cu, Co, Ni, Pt, Pd, Mg, or Ca.

(4) The photoelectric conversion element according to any one of (1) to (3), in which M represents Zn.

(5) The photoelectric conversion element according to any one of (1) to (4), in which a maximum absorption wavelength of the compound represented by Formula (1) described below is within a range of 480 to 600 nm.

(6) The photoelectric conversion element according to any one of (1) to (5), in which in a case where the photoelectric conversion film has a maximum absorption wavelength within a range of 480 to 600 nm and light absorbance of the photoelectric conversion film at the maximum absorption wavelength is 1, each of relative values of the light absorbance of the photoelectric conversion film at 400 nm and at 650 nm is 0.10 or less.

(7) The photoelectric conversion element according to any one of (1) to (6), in which a molecular weight of the compound represented by Formula (1) is 400 to 1200.

(8) The photoelectric conversion element according to any one of (1) to (7), in which the photoelectric conversion film has a bulk hetero structure.

(9) The photoelectric conversion element according to any one of (1) to (8), further including one or more interlayers between the conductive film and the transparent conductive film, in addition to the photoelectric conversion film.

(10) An optical sensor comprising the photoelectric conversion element according to any one of (1) to (9).

(11) An imaging element comprising the photoelectric conversion element according to any one of (1) to (9).

(12) A compound represented by Formula (1-1) described below.

According to the present invention, it is possible to provide a photoelectric conversion element exhibiting excellent responsiveness, and excellent dark current characteristics in a case of high-speed film formation.

Also, according to the present invention, it is possible to provide an optical sensor, an imaging element, and a compound which include the photoelectric conversion element.

### BRIEF DESCRIPTION OF THE DRAWINGS

FIG. 1A is a schematic cross-sectional view showing an example of a configuration of a photoelectric conversion element.

FIG. 1B is a schematic cross-sectional view showing an example of a configuration of a photoelectric conversion element.

FIG. 2 is a schematic cross-sectional view of one pixel of a hybrid type photoelectric conversion element.

FIG. 3 is a schematic cross-sectional view of one pixel of an imaging element.

### DESCRIPTION OF THE PREFERRED EMBODIMENTS

Hereinafter, preferred embodiments of a photoelectric conversion element of the present invention will be described.

In the present specification, a substituent for which whether it is substituted or unsubstituted is not specified may be further substituted with a substituent (preferably, a substituent W described below) within the scope not impairing an intended effect. For example, the expression of “alkyl group” corresponds to an alkyl group with which a substituent (preferably, the substituent W described below) may be substituted.

In addition, in the present specification, the numerical range represented by “to” means a range including numerical values denoted before and after “to” as a lower limit value and an upper limit value.

Also, in the present specification, 1 Å (angstrom) corresponds to 0.1 nm.

An example of a characteristic point of the present invention compared with the technique in the related art includes a point that a compound represented by Formula (1) described below (hereinafter, also simply referred to as a “specific compound”) is used together with at least one selected from the group consisting of a compound represented by Formula (2) described below and a compound represented by Formula (3) described below, as an n-type organic semiconductor.

In the specific compound, because two pyrromethene moieties coordinate to a metal atom, a structure of the specific compound is three-dimensional. Therefore, the specific compound is difficult to be crystallized, and as a result, it is considered that the influence of the vapor deposition rate on the performance is small. Also, the specific compound is considered to be excellent in responsiveness because the charge transport anisotropy is relatively small.

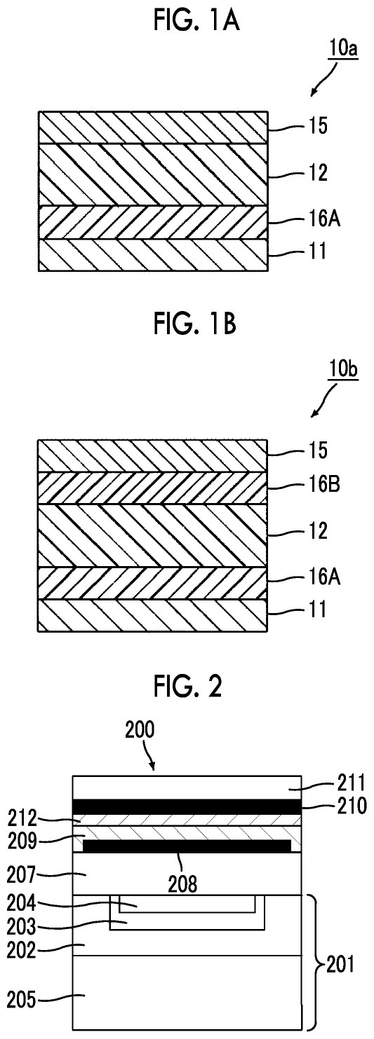

Hereinafter, preferred embodiments of a photoelectric conversion element of the present invention will be described with reference to drawings. A schematic cross-sectional view of an embodiment of a photoelectric conversion element of the present invention is shown in FIGS. 1A and 1B.

A photoelectric conversion element 10a shown in FIG. 1A has a configuration in which a conductive film (hereinafter, also referred to as a lower electrode) 11 functioning as the lower electrode, an electron blocking film 16A, a photoelectric conversion film 12 containing the compound represented by Formula (1) described below, and a transparent conductive film (hereinafter, also referred to as an upper electrode) 15 functioning as the upper electrode are laminated in this order.

FIG. 1B shows a configuration example of another photoelectric conversion element. A photoelectric conversion element 10b shown in FIG. 1B has a configuration in which the electron blocking film 16A, the photoelectric conversion film 12, a positive hole blocking film 16B, and the upper electrode 15 are laminated on the lower electrode 11 in this order. The lamination order of the electron blocking film 16A, the photoelectric conversion film 12, and the positive hole blocking film 16B in FIGS. 1A and 1B may be appropriately changed according to the application and the characteristics.

In the photoelectric conversion element 10a (or 10b), it is preferable that light is incident on the photoelectric conversion film 12 through the upper electrode 15.

Also, in a case where the photoelectric conversion element 10a (or 10b) is used, a voltage can be applied. In this case, the lower electrode 11 and the upper electrode 15 form a pair of electrodes, it is preferable that the voltage of 1×10⁻⁵ to 1×10⁷ V/cm is applied between the pair of electrodes. From the viewpoint of performance and power consumption, the voltage to be applied is more preferably 1×10⁻⁴ to 1×10⁷ V/cm, and still more preferably 1×10⁻³ to 5×10⁶ V/cm.

The voltage application method is preferable that the voltage is applied such that the electron blocking film 16A side is a cathode and the photoelectric conversion film 12 side is an anode, in FIGS. 1A and 1B. In a case where the photoelectric conversion element 10a (or 10b) is used as an optical sensor, or also in a case where the photoelectric conversion element 10a (or 10b) is incorporated in an imaging element, the voltage can be applied by the same method.

As described in detail below, the photoelectric conversion element 10a (or 10b) can be suitably applied to applications of the optical sensor and the imaging element.

In addition, a schematic cross-sectional view of another embodiment of a photoelectric conversion element of the present invention is shown in FIG. 2.

The photoelectric conversion element 200 shown in FIG. 2 is a hybrid type photoelectric conversion element comprising an organic photoelectric conversion film 209 and an inorganic photoelectric conversion film 201. The organic photoelectric conversion film 209 contains the compound represented by Formula (1) described below.

The inorganic photoelectric conversion film 201 has an n-type well 202, a p-type well 203, and an n-type well 204 on a p-type silicon substrate 205.

Blue light is photoelectrically converted (B pixel) at a p-n junction formed between the p-type well 203 and the n-type well 204, and red light is photoelectrically converted (R pixel) at a p-n junction formed between the p-type well 203 and the n-type well 202. The conduction types of the n-type well 202, the p-type well 203, and the n-type well 204 are not limited thereto.

Furthermore, a transparent insulating layer 207 is disposed on the inorganic photoelectric conversion film 201.

A transparent pixel electrode 208 divided for each pixel is disposed on the insulating layer 207. The organic photoelectric conversion film 209 which absorbs green light and performs photoelectric conversion is disposed on the transparent pixel electrode in a single layer configuration commonly for each pixel. The electron blocking film 212 is disposed on the organic photoelectric conversion film in a single layer configuration commonly for each pixel. A transparent common electrode 210 with a single layer configuration is disposed on the electron blocking film. A transparent protective film 211 is disposed on the uppermost layer. The lamination order of the electron blocking film 212 and the organic photoelectric conversion film 209 may be reversed from that in FIG. 2, and the common electrode 210 may be disposed so as to be divided for each pixel.

The organic photoelectric conversion film 209 constitutes a G pixel for detecting green light.

The pixel electrode 208 is the same as the lower electrode 11 of the photoelectric conversion element 10a shown in FIG. 1A. The common electrode 210 is the same as the upper electrode 15 of the photoelectric conversion element 10a shown in FIG. 1A.

In a case where light from a subject is incident on the photoelectric conversion element 200, green light in the incident light is absorbed by the organic photoelectric conversion film 209 to generate optical charges. The optical charges flow into and accumulate in a green signal charge accumulation region not shown in the drawing from the pixel electrode 208.

Mixed light of the blue light and the red light transmitted through the organic photoelectric conversion film 209 enters the inorganic photoelectric conversion film 201. The blue light having a short wavelength is photoelectrically converted mainly at a shallow portion (in the vicinity of the p-n junction formed between the p-type well 203 and the n-type well 204) of a semiconductor substrate (the inorganic photoelectric conversion film) 201 to generate optical charges, and a signal is output to the outside. The red light having a long wavelength is photoelectrically converted mainly at a deep portion (in the vicinity of the p-n junction formed between the p-type well 203 and the n-type well 202) of the semiconductor substrate (the inorganic photoelectric conversion film) 201 to generate optical charges, and a signal is output to the outside.

In a case where the photoelectric conversion element 200 is used in the imaging element, a signal readout circuit (an electric charge transfer path in a case of a charge coupled device (CCD) type, or a metal-oxide-semiconductor (MOS) transistor circuit in a case of a complementary metal oxide semiconductor (CMOS) type), or the green signal charge accumulation region is formed in a surface portion of the p-type silicon substrate 205. In addition, the pixel electrode 208 is connected to the corresponding green signal charge accumulation region through vertical wiring.

Hereinafter, the form of each layer constituting the photoelectric conversion element of the present invention will be described in detail.

### Photoelectric Conversion Film

(Compound Represented by Formula (1)) The photoelectric conversion film 12 (or the organic photoelectric conversion film 209) is a film containing the compound represented by Formula (1) as a photoelectric conversion material. The photoelectric conversion element exhibiting excellent responsiveness, and excellent dark current characteristics in a case of high-speed film formation can be obtained by using the compound.

Hereinafter, the compound represented by Formula (1) will be described in detail.

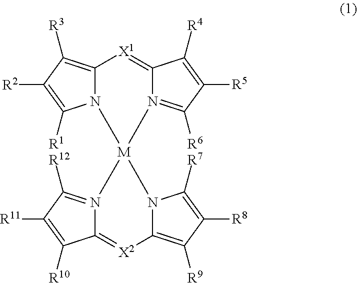

In Formula (1), R¹ to R¹² each independently represent a hydrogen atom or a substituent. The definition of the above-described substituent is synonymous with the substituent W described below.

From the viewpoint of obtaining superior responsiveness and/or the dark current characteristic in a case of high-speed film formation of the photoelectric conversion element (hereinafter, also simply referred to as the “viewpoint of obtaining a superior effect of the present invention”), R¹ to R¹² are each independently preferably a hydrogen atom, a halogen atom, an alkyl group, an aryl group, or a heteroaryl group.

Among these, from the viewpoint of obtaining a superior effect of the present invention, R¹, R³, R⁴, R⁶, R⁷, R⁹, R¹⁰, and R¹² are each independently preferably a hydrogen atom, a halogen atom, an alkyl group, an aryl group, or a heteroaryl group, more preferably a hydrogen atom, an alkyl group, or aryl group, and still more preferably a hydrogen atom, a methyl group, or a phenyl group.

Among these, from the viewpoint of obtaining a superior effect of the present invention, R², R⁵, R⁸, and R¹¹ are preferably a hydrogen atom, an alkyl group, or an aryl group, and more preferably a hydrogen atom.

R¹ and R¹² do not bond to each other to form a ring. R⁶ and R⁷ do not bond to each other to form a ring. R¹ and R² do not bond to each other to form a ring. R⁵ and R⁶ do not bond to each other to form a ring. R⁷ and R⁸ do not bond to each other to form a ring. R¹¹ and R¹² do not bond to each other to form a ring.

In Formula (1), X¹ and X² each independently represent a nitrogen atom, or CR¹³. R¹³ represents a hydrogen atom or a substituent. The definition of the above-described substituent is synonymous with the substituent W described below.

From the viewpoint of obtaining a superior effect of the present invention, X¹ and X² are preferably CR¹³.

Among these, from the viewpoint of obtaining a superior effect of the present invention, R¹³ are preferably a hydrogen atom, an alkyl group, an aryl group, or a heteroaryl group, more preferably a hydrogen atom, an aryl group, or a nitrogen-containing heteroaryl group, and still more preferably a hydrogen atom, a phenyl group, or a nitrogen-containing heteroaryl group.

As described above, an alkyl group, an aryl group, a heteroaryl group, or the like represented by R¹³ may be further substituted with a substituent. Examples of a substituent include the substituent W (for example, an alkyl group, or a halogen atom) described below. Among these, from the viewpoint of obtaining a superior effect of the present invention, the substituent W further included in R¹³ is preferably a fluorine atom or a methyl group.

In Formula (1), M represents a divalent metal atom. Examples of the divalent metal atom represented by M include Zn, Cu, Fe, Co, Ni, Au, Ag, Ir, Ru, Rh, Pd, Pt, Mn, Mg, Ti, Be, Ca, Ba, Cd, Hg, Pb, and Sn. Among these, the divalent metal atom represented by M is preferably Zn, Cu, Co, Ni, Pt, Pd, Mg, or Ca, more preferably Zn, Cu, Co, or Ni, still more preferably Zn, Cu, or Co, and particularly preferably Zn.

The substituent W in the present specification will be described below.

Examples of the substituent W include a halogen atom (a fluorine atom, a chlorine atom, a bromine atom, and an iodine atom, and the like), an alkyl group (including a cycloalkyl group, a bicycloalkyl group, and a tricycloalkyl group), an alkenyl group (including a cycloalkenyl group and a bicycloalkenyl group), an alkynyl group, an aryl group, a heterocyclic group, a cyano group, a hydroxy group, a nitro group, a carboxy group, an alkoxy group, an aryloxy group, a silyloxy group, a heterocyclic oxy group, an acyloxy group, a carbamoyloxy group, an alkoxycarbonyloxy group, an aryloxycarbonyloxy group, an amino group (including an anilino group), an ammonium group, an acylamino group, an aminocarbonylamino group, an alkoxycarbonylamino group, an aryloxycarbonylamino group, a sulfamoylamino group, an alkyl- or arylsulfonylamino group, a mercapto group, an alkylthio group, an arylthio group, a heterocyclic thio group, a sulfamoyl group, a sulfo group, an alkyl- or arylsulfinyl group, an alkyl- or arylsulfonyl group, an acyl group, an aryloxycarbonyl group, an alkoxycarbonyl group, a carbamoyl group, an aryl- or heterocyclic azo group, an imide group, a phosphino group, a phosphinyl group, a phosphinyloxy group, a phosphinylamino group, a phosphono group, a silyl group, a hydrazino group, a ureido group, a boronic acid group (—B(OH)₂), a phosphato group (—OPO(OH)₂), a sulfato group (—OSO₃H), and other well-known substituents.

Moreover, the substituent W may be further substituted by the substituent W. For example, an alkyl group may be substituted with a halogen atom.

The details of the substituent W are disclosed in paragraph [0023] of JP2007-234651A.

The number of carbon atoms in an alkyl group of the specific compound (the compound represented by Formula (1)) is preferably 1 to 10, more preferably 1 to 6, and still more preferably 1 to 4. The alkyl group may be any of linear, branched, or cyclic. Also, the alkyl group may be substituted with a substituent (preferably, the substituent W).

Examples of the alkyl group include a methyl group, an ethyl group, an n-propyl group, an i-propyl group, an n-butyl group, a t-butyl group, an n-hexyl group, and a cyclohexyl group.

The number of carbon atoms in the aryl group of the specific compound (the compound represented by Formula (1)) is not particularly limited, but is preferably 6 to 30 carbon atoms, more preferably 6 to 18 carbon atoms, and still more preferably 6 carbon atoms from the viewpoint of obtaining a superior effect of the present invention. The aryl group may have a monocyclic structure or a condensed ring structure (a fused ring structure) in which two or more rings are condensed. Also, the aryl group may be substituted with a substituent (preferably, the substituent W).

Examples of the aryl group include a phenyl group, a naphthyl group, an anthryl group, a pyrenyl group, a phenanthrenyl group, a methylphenyl group, a dimethylphenyl group, a biphenyl group, and a fluorenyl group, and a phenyl group, a naphthyl group, or an anthryl group is preferable.

The number of carbon atoms in the heteroaryl group (a monovalent aromatic heterocyclic group) of the specific compound (the compound represented by Formula (1)) is not particularly limited, but is preferably 3 to 30 carbon atoms, and more preferably 3 to 18 carbon atoms from the viewpoint of obtaining a superior effect of the present invention. Also, the heteroaryl group may be substituted with a substituent (preferably, the substituent W).

The heteroaryl group includes a hetero atom in addition to a carbon atom and a hydrogen atom. Examples of the hetero atom include a nitrogen atom, a sulfur atom, an oxygen atom, a selenium atom, a tellurium atom, a phosphorus atom, a silicon atom, and a boron atom, and a nitrogen atom, a sulfur atom, or an oxygen atom is preferable.

The number of hetero atoms contained in the heteroaryl group is not particularly limited, but is usually about 1 to 10, preferably 1 to 4, and more preferably 1 to 2.

The number of ring members of the heteroaryl group is not particularly limited, but is preferably 3 to 8, more preferably 5 to 7, and still more preferably 5 to 6. The heteroaryl group may have a monocyclic structure or a condensed ring structure in which two or more rings are condensed. In a case of the condensed ring structure, an aromatic hydrocarbon ring having no hetero atom (for example, a benzene ring) may be included.

Examples of the heteroaryl group include a pyridyl group, a quinolyl group, an isoquinolyl group, an acridinyl group, a phenanthridinyl group, a pteridinyl group, a pyrazinyl group, a quinoxalinyl group, a pyrimidinyl group, a quinazolyl group, a pyridazinyl group, a cinnolinyl group, a phthalazinyl group, a triazinyl group, an oxazolyl group, a benzoxazolyl group, a thiazolyl group, a benzothiazolyl group, an imidazolyl group, a benzimidazolyl group, a pyrazolyl group, an indazolyl group, an isoxazolyl group, a benzisoxazolyl group, an isothiazolyl group, a benzisothiazolyl group, an oxadiazolyl group, a thiadiazolyl group, a triazolyl group, a tetrazolyl group, a furyl group, a benzofuryl group, a thienyl group, a benzothienyl group, a dibenzofuryl group, a dibenzothienyl group, a pyrrolyl group, an indolyl group, an imidazopyridinyl group, and a carbazolyl group.

One of the preferred aspects of the compound represented by the formula (1) is a compound represented by the formula (1-1).

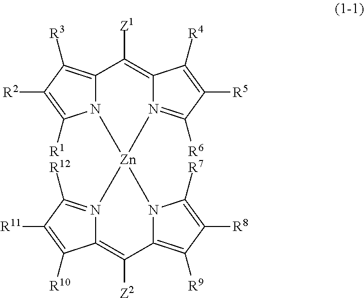

In Formula (1-1), R¹ to R¹² each independently represent a hydrogen atom, an alkyl group, an aryl group, or a heteroaryl group. The definitions of the alkyl group, the aryl group, and the heteroaryl group are as described above.

In Formula (1-1), R¹, RR³, R⁴, R⁶, R⁷, R⁹, R¹⁰, and R¹² are preferably an alkyl group, an aryl group, or a heteroaryl group.

Among these, R¹ to R¹² are more preferably an alkyl group.

Z¹ and Z² each independently represent an aryl group which has a Hammett substituent constant σₚ exceeding 0 and may have a substituent, or a heteroaryl group which has a Hammett substituent constant σₚ exceeding 0 and may have a substituent.

The aryl group which may have the substituent need only have a Hammett substituent constant σₚ exceeding 0 in the entire group. For example, in a case where the aryl group has a substituent, the Hammett substituent constant σₚ need to exceed 0 in the entire aryl group having a substituent.

Also, the heteroaryl group which may have the substituent need only have the Hammett substituent constant σₚ exceeding 0 in the entire group. For example, in a case where the heteroaryl group has a substituent, the Hammett substituent constant σₚ need to exceed 0 in the entire heteroaryl group having a substituent.

Therefore, in other words, Z¹ and Z² each independently represent an unsubstituted aryl group having the Hammett substituent constant σₚ exceeding 0, an aryl group having a substituent and the Hammett substituent constant σₚ exceeding 0, an unsubstituted heteroaryl group having the Hammett substituent constant σₚ exceeding 0, or a heteroaryl group having a substituent and the Hammett substituent constant σₚ exceeding 0.

Here, the Hammett substituent constant σₚ value will be described. Hammett's rule is a rule of thumb which has been proposed by L. P. Hammett in 1935 in order to quantitatively discuss the influence of substituents on the reaction or equilibrium of benzene derivatives, and is widely accepted today. Substituent constants obtained by Hammett's rule include σₚ value and σₘ value. These values can be found in many pieces of general literature, for example, the values are described in detail in J. A. Dean edition, “Lange's Hand book of Chemistry”, 12th Edition, 1979 (McGraw-Hill), or “Area of Chemistry” supplement, No. 122, pp. 96-103, 1979 (Nankodo). In the present invention, a substituent is limited or described by the Hammett substituent constant σₚ, but it does not mean that it is limited only to a substituent having a known value found in the literature described above. Even the value is unknown in the literature, it also includes substituents to be fallen within the range in a case where measurement is performed based on the Hammett's law.

The definition of the aryl group is as described above, and a phenyl group is preferable.

The kind of substituents that the aryl group may have is not particularly limited, but the substituent W described below is exemplified.

The kind of aryl group which may have the substituent is not particularly limited as long as the Hammett substituent constant σₚ exceeds 0 in the entire group as described above, but it is preferable that the aryl group has an electron attractive group in which the Hammett substituent constant σₚ exceeds 0, as the substituent.

Specific examples of the electron attractive group in which the Hammett substituent constant σₚ exceeds 0 include a halogen atom (a fluorine atom, a chlorine atom, and an iodine atom), a cyano group, a nitro group, and a halogen-substituted alkyl group. Among these, a halogen atom (a fluorine atom, a chlorine atom, and an iodine atom), a cyano group, and a halogen-substituted alkyl group are preferable in that the maximum absorption wavelength of the specific compound tends to be longer.

The number of the electron attractive group included in the aryl group in which the Hammett substituent constant uₚ exceeds 0 is not particularly limited, the number thereof is preferably 1 to 5 in that the maximum absorption wavelength of the specific compound tends to be longer.

The definition of the heteroaryl group is as described above.

Among these, the heteroaryl group is preferably a nitrogen-containing heteroaryl group (a nitrogen-containing aromatic group) in that the maximum absorption wavelength of the specific compound tends to be longer. The nitrogen-containing heteroaryl group is preferably a monocyclic structure.

Examples of the nitrogen-containing heteroaryl group include a pyridyl group, a pyrimidyl group, a pyrazyl group, a triazyl group, a quinolyl group, an imidazolyl group, a pyrazolyl group, a triazyl group, a thiazolyl group, and an oxazolyl group.

The heteroaryl group may have a substituent, and the kind of the substituent is not particularly limited, but the substituent W described below is exemplified. The substituent may be the electron attractive group in which the above-described Hammett substituent constant σₚ exceeds 0.

Hereinafter, the compound represented by Formula (1) will be exemplified.

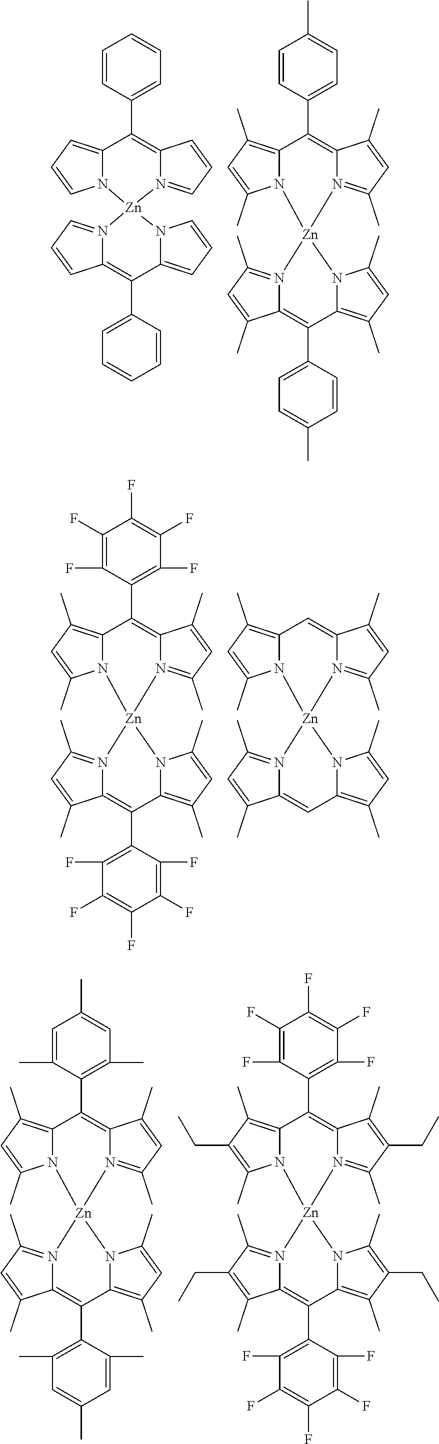

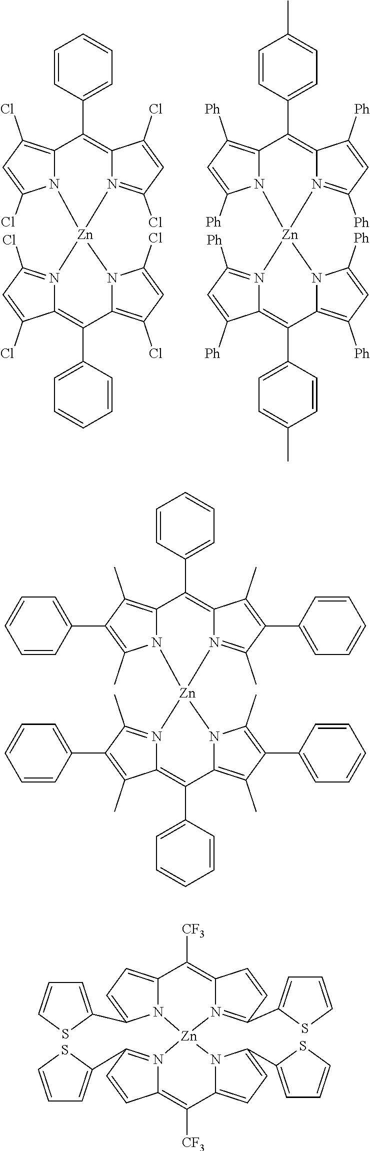

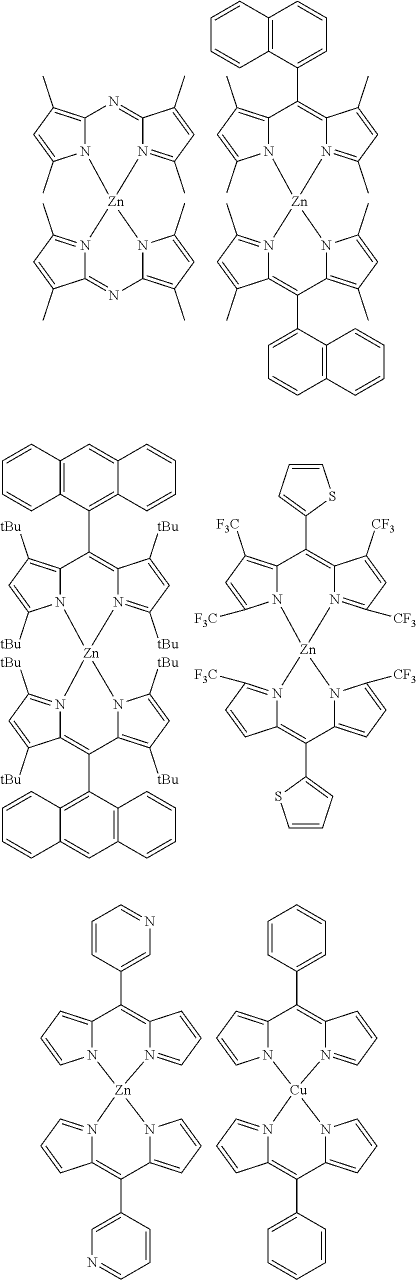

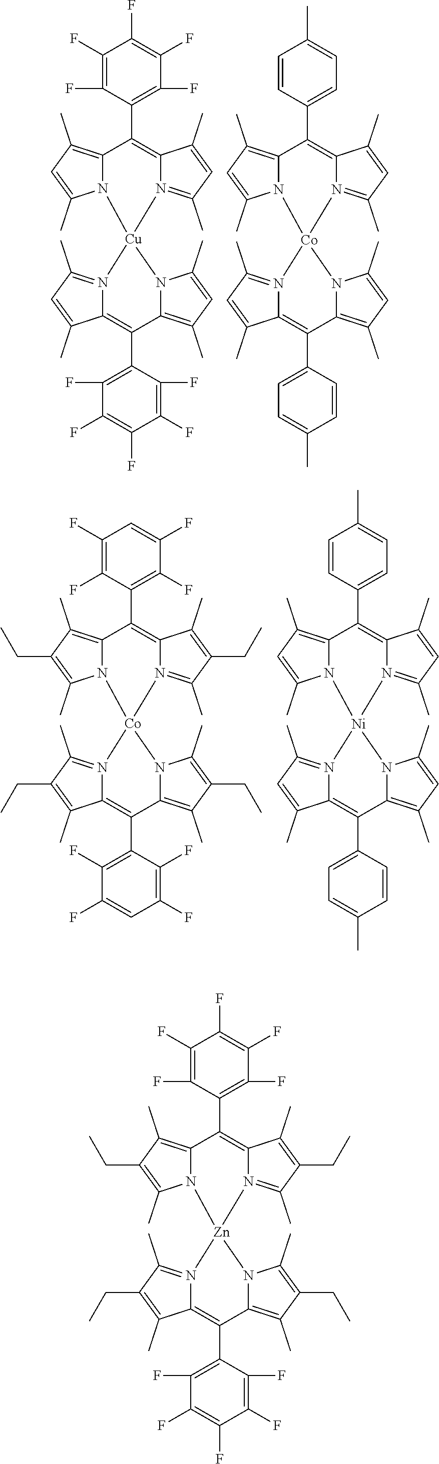

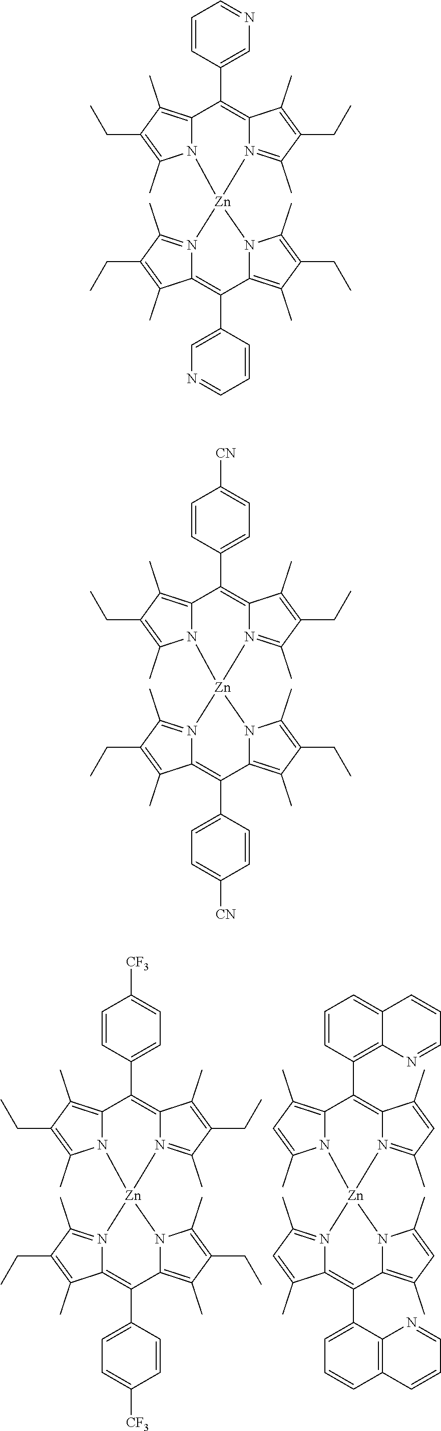

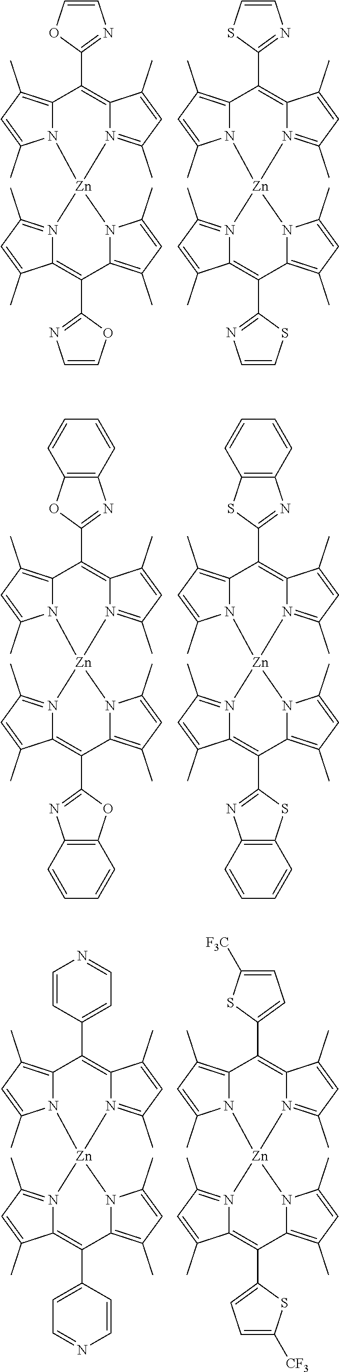

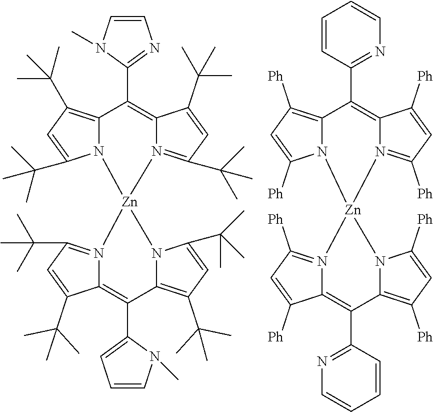

A molecular weight of the compound represented by Formula (1) is not particularly limited, but is preferably 400 to 1200. In a case where the molecular weight is 1200 or less, the vapor deposition temperature does not increase, and the decomposition of the compound hardly occurs. In a case where the molecular weight is 400 or more, a glass transition point of a deposited film does not decrease, and a heat resistance of the photoelectric conversion element is improved.

In order to be applicable to the organic photoelectric conversion film 209 that absorbs green light and performs photoelectric conversion as described above, the maximum absorption wavelength of the compound represented by Formula (1) is preferably in a range of 450 to 600 nm, and more preferably in a range of 480 to 600 nm.

The maximum absorption wavelength is a value measured in a solution state (a solvent is chloroform) by adjusting the absorption spectrum of the compound represented by the formula (1) to a concentration at which the light absorbance is 0.5 to 1.

The compound represented by Formula (1) is preferably a compound in which an ionization potential in a single film is −5.0 to −6.0 eV from the viewpoints of stability in a case of using the compound as the p-type organic semiconductor and matching of energy levels between the compound and the n-type organic semiconductor.

The compound represented by Formula (1) is particularly useful as a material of the photoelectric conversion film used for the optical sensor, the imaging element, or a photoelectric cell. In addition, the compound represented by Formula (1) usually functions as the p-type organic semiconductor in the photoelectric conversion film in many cases. The compound represented by the formula (1) can also be used as a coloring material, a liquid crystal material, an organic semiconductor material, a charge transport material, a pharmaceutical material, and a fluorescent diagnostic material.

(n-Type Organic Semiconductor)

The photoelectric conversion film contains the n-type organic semiconductor as a component other than the compound represented by the above-mentioned Formula (1).

The n-type organic semiconductor is an acceptor-property organic semiconductor material (a compound), and refers to an organic compound having a property of easily accepting an electron. More specifically, in the present specification, the n-type organic semiconductor refers to an organic compound having a larger electron affinity than the compound represented by Formula (1) when compared with the compound represented by Formula (1).

The n-type organic semiconductor contains at least one selected from the group consisting of the compound represented by Formula (2) and the compound represented by Formula (3), and it is preferable that the n-type organic semiconductor contains the compound represented by Formula (3) from the viewpoint of obtaining a superior effect of the present invention.

First, the compound represented by Formula (2) will be described in detail.

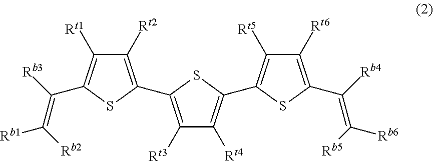

In Formula (2), R^{t1} to R^{t6} each independently represent a hydrogen atom or a substituent. The definition of the above-described substituent is synonymous with the substituent W described above.

Among these, from the viewpoint of obtaining a superior effect of the present invention, R^{t1}, R^{t2}, R^{t3}, and R^{t4} are each independently preferably a hydrogen atom, an alkyl group, an aryl group, or a heteroaryl group, more preferably a hydrogen atom, or an alkyl group, and still more preferably a hydrogen atom. Here, the preferred range of the alkyl group, the aryl group, or the heteroaryl group represented by R^{t1}, R^{t2}, R^{t5}, and R^{t6} is the same as the preferred range of an alkyl group, an aryl group, or a heteroaryl group which is included in the compound represented by Formula (1) as a substituent.

Among these, from the viewpoint of obtaining a superior effect of the present invention, R^{t3} and R^{t4} are each independently preferably a hydrogen atom, an alkyl group, an aryl group, or a heteroaryl group, more preferably a hydrogen atom or an alkyl group, still more preferably a linear or branched alkyl group having 2 to 8 carbon atoms, and particularly preferably a linear alkyl group having 4 to 6 carbon atoms.

Here, the preferred range of the aryl group or the heteroaryl group represented by R^{t3} and R^{t4} is the same as the preferred range of an aryl group or a heteroaryl group which is included in the compound represented by Formula (1) as a substituent.

In Formula (2), R^{b1} to R^{b6} each independently represent a hydrogen atom or a substituent. The definition of the above-described substituent is synonymous with the substituent W described above.

Also, at least one of R^{b1}, . . . , or R^{b6} represents an electron attractive group.

As the electron attractive group represented by R^{b1} to R^{b6}, a halogen atom, a halogenated alkyl group, a halogenated aryl group, a halogenated heteroaryl group, a nitrogen-containing heteroaryl group, a methyl ester group, a cyano group, a nitro group, a carbonyl group, a sulfonyl group, a phosphoryl group, and an alkynyl group are exemplified. Among these, from the viewpoint of obtaining a superior effect of the present invention, the electron attractive group represented by R^{b1} to R^{b6} is preferably a halogenated alkyl group, a methyl ester group, and a cyano group, and more preferably a cyano group.

In a case where a plurality of electron attractive groups represented by R^{b1} to R^{b6} are present, the kinds of the plurality of electron attractive group may be different from each other. The number of the electron attractive groups represented by R^{b1} to R^{b6} is preferably 2 to 6, and more preferably 2 to 4.

From the viewpoint of obtaining a superior effect of the present invention, among R^{b1} to R^{b6}, it is preferable that R^{b1}, R^{b2}, R^{b5}, and R^{b6} are the electron attractive group. R^{b3} and R^{b4} are preferably a group other than the electron attractive group, and more preferably a hydrogen atom.

Hereinafter, the compound represented by Formula (2) will be exemplified.

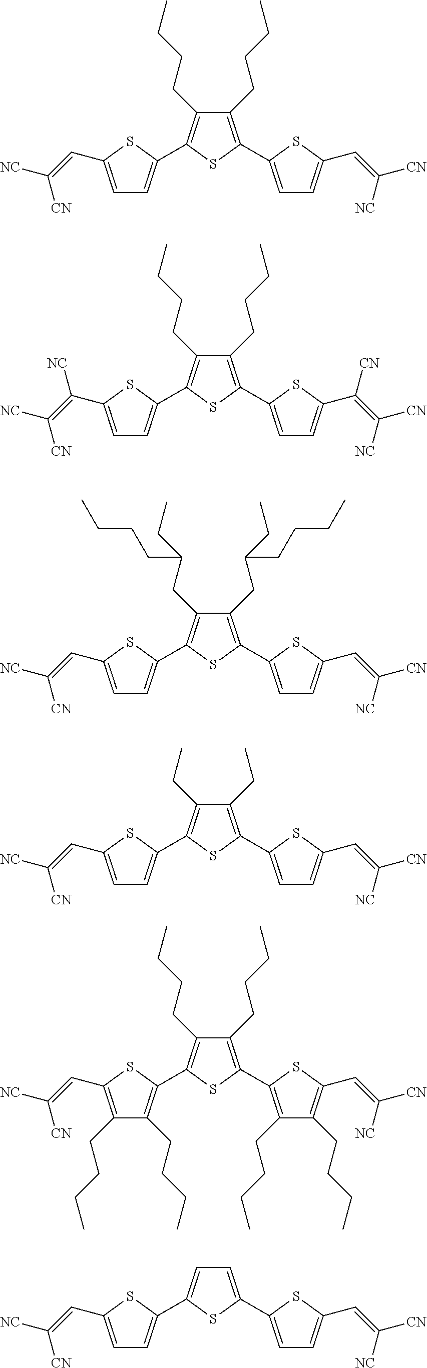

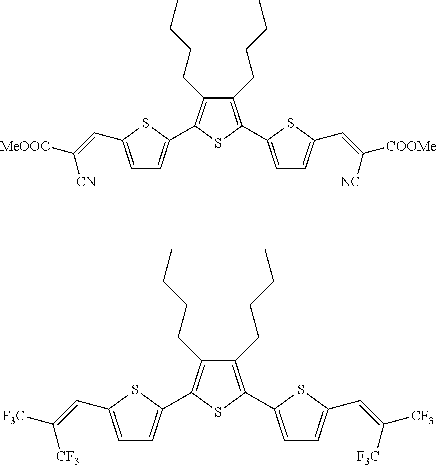 [SMILES: CCCCc1c(-c2ccc(/C=C(\C#N)C(=O)OC)s2)sc(-c2ccc(/C=C(\C#N)C(=O)OC)s2)c1CCCC ; CCCCc1c(-c2ccc(C=C(C(F)(F)F)C(F)(F)F)s2)sc(-c2ccc(C=C(C(F)(F)F)C(F)(F)F)s2)c1CCCC]

Next, the compound represented by Formula (3) will be described in detail.

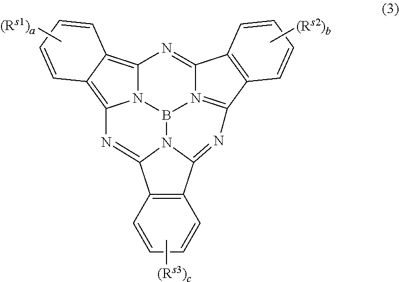

In Formula (3), R^{s1} to R^{s3} each independently represent a substituent. The definition of the above-described substituent is synonymous with the substituent W described above.

Among these, from the viewpoint of obtaining a superior effect of the present invention, R^{s1} to R^{s3} are each independently preferably a halogen atom, an alkyl group, an aryl group, or a heteroaryl group, more preferably a halogen atom, and still more preferably a fluorine atom.

Here, the preferred range of the alkyl group, the aryl group, or the heteroaryl group represented by R^{s1} to R^{s3} is the same as the preferred range of an alkyl group, an aryl group, or a heteroaryl group that the compound represented by Formula (1) has as a substituent.

In Formula (3), a to c each independently represent an integer of 0 to 4.

The integers represented by a to c are each independently preferably 1 to 4, and more preferably 2 to 4.

In a case where a represents an integer of 2 or more, the plurality of R^{s1} may be different from each other, in a case where b represents an integer of 2 or more, the plurality of R^{s2} may be different from each other, and in a case where c represents an integer of 2 or more, the plurality of R^{s3} may be different from each other.

In Formula (3), Y¹ represents a halogen atom, an alkoxy group, an aryloxy group, an alkylthio group, an arylthio group, a group having a carbonyloxy group (preferably, a group represented by R^{Y}—CO—O—, R^{Y} represents a hydrogen atom or a substituent (for example, a substituent W)), an amino group, an ethynyl group, or an ethenyl group. Among these, from the viewpoint of obtaining a superior effect of the present invention, Y¹ is preferably an aryloxy group or a halogen atom, and more preferably a halogen atom.

As described above, in a case where a group represented by Y¹ further has a substituent, a group represented by Y¹ may be substituted with a substituent. As the substituent, the above-described substituent W (for example, a halogen atom) is exemplified.

Hereinafter, the compound represented by Formula (3) will be exemplified.

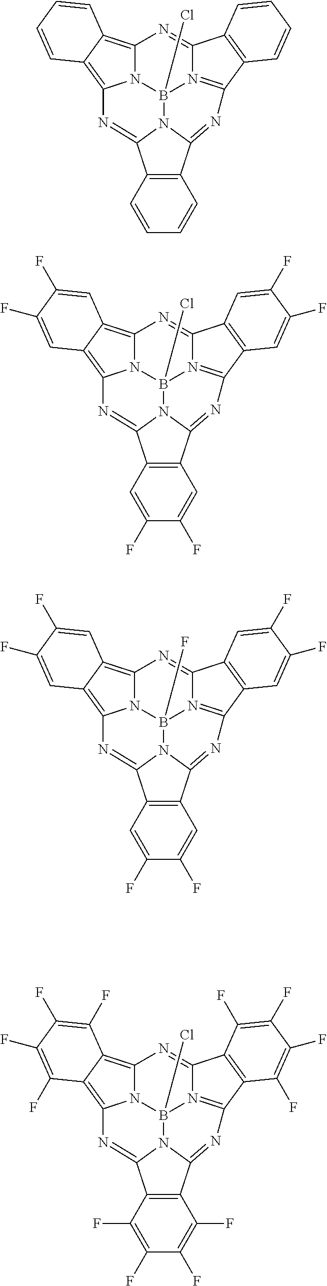

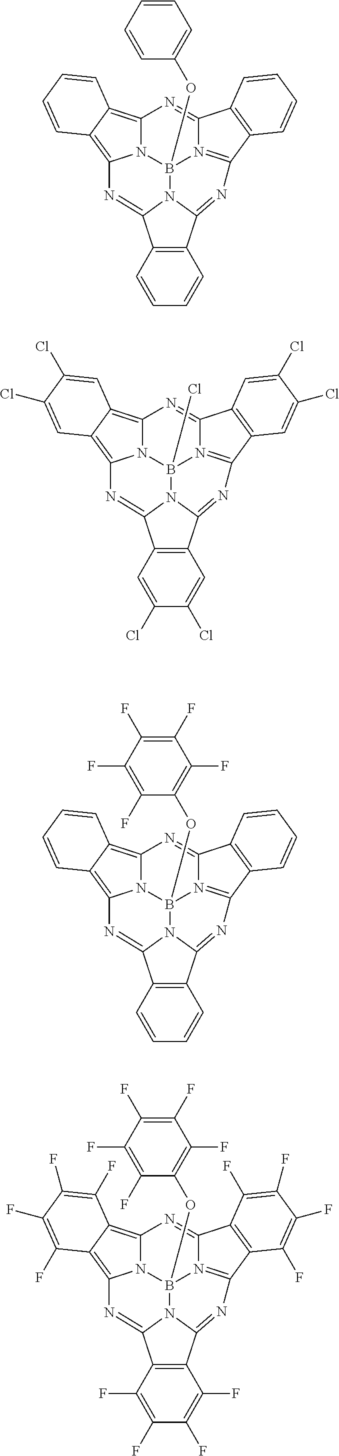

In the case of the form as shown in FIG. 2, it is preferable that the specific n-type organic semiconductor is colorless, or has a maximum absorption wavelength and/or an absorption waveform close to the compound represented by Formula (1). Specifically, the maximum absorption wavelength of the n-type organic semiconductor is preferably 500 to 600 nm from the viewpoint of obtaining a superior effect of the present invention.

The photoelectric conversion film may contain components other than the compounds represented by Formulae (1) to (3) described above. For example, the photoelectric conversion film may contain the n-type organic semiconductor other than the compound represented by Formula (2) and the compound represented by Formula (3).

The maximum absorption wavelength of the photoelectric conversion film is preferably in a range of 450 to 600 nm, and more preferably in a range of 480 to 600 nm in order to be applicable to the organic photoelectric conversion film 209 that absorbs green light and performs photoelectric conversion as described above.

Moreover, from the viewpoint of obtaining a superior effect of the present invention, in a case where the photoelectric conversion film has the maximum absorption wavelength in a range of 480 to 600 nm, and in a case where the light absorbance in the maximum absorption wavelength is 1, it is preferable that each of the relative values of the light absorbance of the photoelectric conversion film at 400 nm and at 650 nm is 0.10 or less.

The light absorbance of the photoelectric conversion film is measured by using a spectrophotometer UV-3600 manufactured by Shimadzu Corporation. Specifically, a film is produced on a 2.5 cm square glass substrate, the substrate is fixed to a film holder attached to the spectrophotometer, and the transmittance is measured to obtain the light absorbance.

It is preferable that the photoelectric conversion film has a bulk hetero structure formed in a state in which the compound represented by Formula (1) and the n-type organic semiconductor are mixed. The bulk hetero structure refers to a layer in which the compound represented by Formula (1) and the n-type organic semiconductor are mixed and dispersed in the photoelectric conversion film. The photoelectric conversion film having the bulk hetero structure can be formed by either a wet method or a dry method. The bulk hetero structure is described in detail in, for example, paragraphs [0013] to [0014] of JP2005-303266A.

The content of the compound represented by Formula (1) to the total content of the compound represented by Formula (1) and the n-type organic semiconductor (=film thickness in terms of single layer of compound represented by Formula (1)/(film thickness in terms of single layer of compound represented by Formula (1) +film thickness in terms of single layer of n-type organic semiconductor)×100) is preferably 20 to 80 volume %, more preferably 30 to 70 volume %, and still more preferably 40 to 60 volume % from the viewpoint of responsiveness of the photoelectric conversion element.

It is preferable that the photoelectric conversion film is substantially formed of the compound represented by Formula (1) and the n-type organic semiconductor. The term of “substantially” means that the total content of the compound represented by Formula (1) and the n-type organic semiconductor to the total mass of the photoelectric conversion film is 95 mass % or more.

The photoelectric conversion film containing the compound represented by Formula (1) is a non-luminescent film, and has a feature different from an organic light emitting diode (OLED). The non-luminescent film means a film having a luminescence quantum efficiency of 1% or less, and the luminescence quantum efficiency is preferably 0.5% or less, and more preferably 0.1% or less.

(Film Formation Method)

The photoelectric conversion film can be formed mostly by a dry film formation method. Specific examples of the dry film formation method include a physical vapor deposition method such as a vapor deposition method (in particular, a vacuum evaporation method), a sputtering method, an ion plating method, and molecular beam epitaxy (MBE), and chemical vapor deposition (CVD) such as plasma polymerization. Among these, the vacuum evaporation method is preferable. In a case where the photoelectric conversion film is formed by the vacuum evaporation method, a producing condition such as a degree of vacuum and a vapor deposition temperature can be set according to the normal method.

The thickness of the photoelectric conversion film is preferably 10 to 1000 nm, more preferably 50 to 800 nm, and still more preferably 100 to 500 nm.

### Electrode

The electrode (the upper electrode (the transparent conductive film) 15 and the lower electrode (the conductive film) 11) is formed of a conductive material. Examples of the conductive material include metals, alloys, metal oxides, electrically conductive compounds, and mixtures thereof.

Since light is incident through the upper electrode 15, the upper electrode 15 is preferably transparent to light to be detected. Examples of the material forming the upper electrode 15 include conductive metal oxides such as tin oxide (ATO, FTO) doped with antimony, fluorine, or the like, tin oxide, zinc oxide, indium oxide, indium tin oxide (ITO), and indium zinc oxide (IZO); metal thin films such as gold, silver, chromium, and nickel, mixtures or laminates of these metals and the conductive metal oxides; and organic conductive materials such as polyaniline, polythiophene, and polypyrrole. Among these, conductive metal oxides are preferable from the viewpoints of high conductivity, transparency, and the like.

In general, in a case where the conductive film is made to be thinner than a certain range, a resistance value is rapidly increased. However, in the solid-state imaging element into which the photoelectric conversion element according to the present embodiment is incorporated, the sheet resistance is preferably 100 to 10000 Ω/□, and the degree of freedom of the range of the film thickness that can be thinned is large. In addition, as the thickness of the upper electrode (the transparent conductive film) 15 is thinner, the amount of light that the upper electrode absorbs becomes smaller, and the light transmittance usually increases. The increase in the light transmittance causes an increase in light absorbance in the photoelectric conversion film and an increase in the photoelectric conversion ability, which is preferable. Considering the suppression of leakage current, an increase in the resistance value of the thin film, and an increase in transmittance accompanied by the thinning, the film thickness of the upper electrode 15 is preferably 5 to 100 nm, and more preferably 5 to 20 nm.

There is a case where the lower electrode 11 has transparency, or an opposite case where the lower electrode does not have transparency and reflects light, depending on the application. Examples of a material constituting the lower electrode 11 include conductive metal oxides such as tin oxide (ATO, FTO) doped with antimony, fluorine, or the like, tin oxide, zinc oxide, indium oxide, indium tin oxide (ITO), and indium zinc oxide (IZO); metals such as gold, silver, chromium, nickel, titanium, tungsten, and aluminum, conductive compounds (for example, titanium nitride (TiN)) such as oxides or nitrides of these metals; mixtures or laminates of these metals and conductive metal oxides; and organic conductive materials such as polyaniline, polythiophene, and polypyrrole.

The method of forming electrodes is not particularly limited, and can be appropriately selected in accordance with the electrode material. Specific examples thereof include a wet method such as a printing method and a coating method; a physical method such as a vacuum evaporation method, a sputtering method, and an ion plating method; and a chemical method such as a CVD method and a plasma CVD method.

In a case where the material of the electrode is ITO, examples thereof include an electron beam method, a sputtering method, a resistance thermal vapor deposition method, a chemical reaction method (such as a sol-gel method), and a coating method with a dispersion of indium tin oxide.

### Charge Blocking Film: Electron Blocking Film and Positive Hole Blocking Film

It is also preferable that the photoelectric conversion element of the present invention has one or more interlayers between the conductive film and the transparent conductive film, in addition to the photoelectric conversion film. Example of the interlayer includes the charge blocking film. In the case where the photoelectric conversion element has this film, the characteristics (such as photoelectric conversion efficiency and responsiveness) of the photoelectric conversion element to be obtained become superior. Examples of the charge blocking film include the electron blocking film and the positive hole blocking film. Hereinafter, the films will be described in detail.

(Electron Blocking Film)

The electron blocking film includes an electron donating compound. Specific examples of a low molecular material include aromatic diamine compounds such as N,N′-bis(3-methylphenyl)-(1,1′-biphenyl)-4,4′-diamine (TPD) and 4,4′-bis[N-(naphthyl)-N-phenyl-amino]biphenyl (α-NPD); porphyrin compounds such as porphyrin, copper tetraphenylporphyrin, phthalocyanine, copper phthalocyanine, and titanium phthalocyanine oxide; oxazole, oxadiazole, triazole, imidazole, imidazolone, a stilbene derivative, a pyrazoline derivative, tetrahydroimidazole, polyarylalkane, butadiene, 4,4′,4″-tris(N-(3-methylphenyl)-N-phenylamino) triphenylamine (m-MTDATA), a triazole derivative, an oxadiazole derivative, an imidazole derivative, a polyarylalkane derivative, a pyrazoline derivative, a pyrazolone derivative, a phenylenediamine derivative, an arylamine derivative, an amino-substituted chalcone derivative, an oxazole derivative, a styrylanthracene derivative, a fluorenone derivative, a hydrazone derivative, and a silazane derivative. Specific examples of a polymer material include a polymer such as phenylenevinylene, fluorene, carbazole, indole, pyrene, pyrrole, picoline, thiophene, acetylene, and diacetylene, or a derivative thereof. In addition, compounds described in paragraphs [004] to [0063] of JP5597450B, compounds described in paragraphs 0119 to 0158 of JP2011-225544A, and compounds described in paragraphs [0086] to [0090] of JP2012-094660A are exemplified.

The electron blocking film may be configured by a plurality of films. The electron blocking film may be formed of an inorganic material. In general, an inorganic material has a dielectric constant larger than that of an organic material. Therefore, in a case where the inorganic material is used in the electron blocking film, a large voltage is applied to the photoelectric conversion film. Therefore, the photoelectric conversion efficiency increases. Examples of the inorganic material that can be used in the electron blocking film include calcium oxide, chromium oxide, copper chromium oxide, manganese oxide, cobalt oxide, nickel oxide, copper oxide, copper gallium oxide, copper strontium oxide, niobium oxide, molybdenum oxide, copper indium oxide, silver indium oxide, and iridium oxide.

(Positive Hole Blocking Film)

The positive hole blocking film includes an electron accepting compound.

Examples of the electron accepting compound include an oxadiazole derivative such as 1,3-bis(4-tert-butylphenyl-1,3,4-oxadiazolyl)phenylene (OXD-7); an anthraquinodimethane derivative; a diphenylquinone derivative; bathocuproine, bathophenanthroline, and derivatives thereof; a triazole compound; a tris(8-hydroxyquinolinato)aluminum complex; a bis(4-methyl-8-quinolinato)aluminum complex; a distyrylarylene derivative; and a silole compound. In addition, compounds described in paragraphs [0056] to [0057] of JP2006-100767A are exemplified.

The method of producing the charge blocking film is not particularly limited, a dry film formation method and a wet film formation method are exemplified. Examples of the dry film formation method include a vapor deposition method and a sputtering method. The vapor deposition method may be any of physical vapor deposition (PVD) and chemical vapor deposition (CVD), and physical vapor deposition such as vacuum evaporation method is preferable. Examples of the wet film formation method include an inkjet method, a spray method, a nozzle printing method, a spin coating method, a dip coating method, a casting method, a die coating method, a roll coating method, a bar coating method, and a gravure coating method, and an inkjet method is preferable from the viewpoint of high precision patterning.

Each thickness of the charge blocking films (the electron blocking film and the positive hole blocking film) is preferably 10 to 200 nm, more preferably 30 to 150 nm, and still more preferably 50 to 100 nm.

### Substrate

The photoelectric conversion element may further include a substrate. The type of substrate to be used is not particularly limited, and a semiconductor substrate, a glass substrate, and a plastic substrate are exemplified.

The position of the substrate is not particularly limited, but in general, the conductive film, the photoelectric conversion film, and the transparent conductive film are laminated on the substrate in this order.

### Sealing Layer

The photoelectric conversion element may further include a sealing layer. The performance of the photoelectric conversion material may deteriorate noticeably due to the presence of deterioration factors such as water molecules. The deterioration can be prevented by sealing and coating the entirety of the photoelectric conversion film with the sealing layer such as diamond-like carbon (DLC) or ceramics such as metal oxide, metal nitride, and metal nitride oxide which are dense and into which water molecules do not permeate.

The material of the sealing layer may be selected and the sealing layer may be produced according to the description in paragraphs [0210] to [0215] of JP2011-082508A.

### Optical Sensor

Examples of the application of the photoelectric conversion element include the photoelectric cell and the optical sensor, but the photoelectric conversion element of the present invention is preferably used as the optical sensor. The photoelectric conversion element may be used alone as the optical sensor. Alternately, the photoelectric conversion element may be used as a line sensor in which the photoelectric conversion elements are linearly arranged or as a two-dimensional sensor in which the photoelectric conversion elements are planarly arranged. In the line sensor, the photoelectric conversion element of the present invention functions as the imaging element by converting optical image information into an electric signal using an optical system such as a scanner, and a driving unit. In the two-dimensional sensor, the photoelectric conversion element of the present invention functions as the imaging element by converting the optical image information into the electric signal by imaging the optical image information on the sensor using the optical system such as an imaging module.

### Imaging Element

Next, a configuration example of an imaging element comprising the photoelectric conversion element 10a will be described.

In the configuration example which will be described below, the same reference numerals or the corresponding reference numerals are attached to members or the like having the same configuration or action as those which have already been described, to simplify or omit the description.

The imaging element is an element that converts optical information of an image into the electric signal, and is an element in which a plurality of photoelectric conversion elements are arranged on a matrix in the same plane, optical signals are converted into electric signals in each photoelectric conversion element (pixel), and the electric signals can be sequentially output to the outside of the imaging elements for each pixel. For this reason, one pixel is formed of one photoelectric conversion element and one or more transistors.

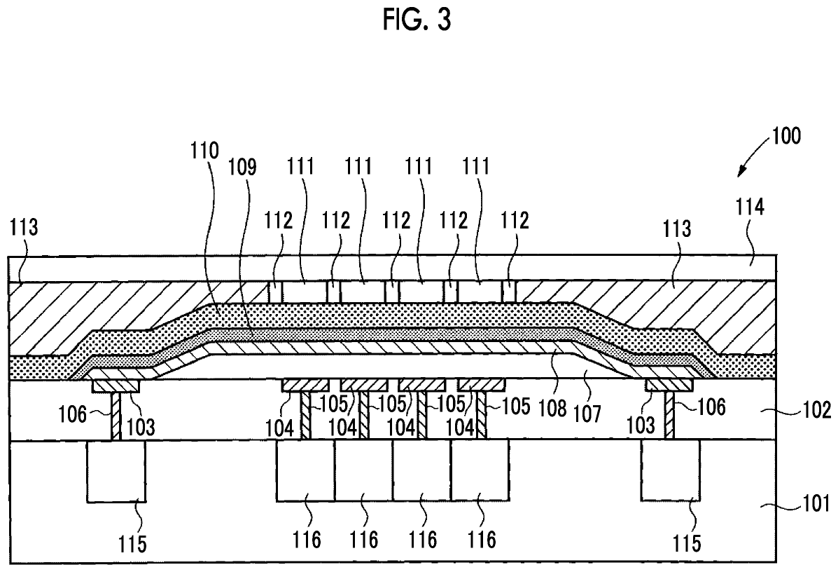

FIG. 3 is a schematic cross-sectional view showing a schematic configuration of an imaging element for describing an embodiment of the present invention. This imaging element is mounted on an imaging device such as a digital camera and a digital video camera, and imaging modules such as an electronic endoscope and a cellular phone.

The imaging element has a plurality of photoelectric conversion elements having configurations shown in FIG. 1A and a circuit substrate in which the readout circuit reading out signals corresponding to charges generated in the photoelectric conversion film of each photoelectric conversion element is formed. The imaging element has a configuration in which the plurality of photoelectric conversion elements are one-dimensionally or two-dimensionally arranged on the same surface above the circuit substrate.

An imaging element 100 shown in FIG. 3 comprises a substrate 101, an insulating layer 102, connection electrodes 103, pixel electrodes (lower electrodes) 104, connection units 105, connection units 106, a photoelectric conversion film 107, a counter electrode (upper electrode) 108, a buffer layer 109, a sealing layer 110, a color filter (CF) 11, partition walls 112, a light shielding layer 113, a protective layer 114, a counter electrode voltage supply unit 115, and readout circuits 116.

The pixel electrode 104 has the same function as the lower electrode 11 of the photoelectric conversion element 10a shown in FIG. 1A. The counter electrode 108 has the same function as the upper electrode 15 of the photoelectric conversion element 10a shown in FIG. 1A. The photoelectric conversion film 107 has the same configuration as a layer provided between the lower electrode 11 and the upper electrode 15 of the photoelectric conversion element 10a shown in FIG. 1A.

The substrate 101 is a semiconductor substrate such as the glass substrate, or Si. The insulating layer 102 is formed on the substrate 101. A plurality of pixel electrodes 104 and a plurality of connection electrodes 103 are formed on the surface of the insulating layer 102.

The photoelectric conversion film 107 is a layer common to all the photoelectric conversion elements provided so as to cover the plurality of pixel electrodes 104.

The counter electrode 108 is one electrode common to all the photoelectric conversion elements provided on the photoelectric conversion film 107. The counter electrode 108 is formed on the connection electrodes 103 arranged on an outer side than the photoelectric conversion film 107, and is electrically connected to the connection electrodes 103.

The connection units 106 are buried in the insulating layer 102, and are plugs for electrically connecting the connection electrodes 103 to the counter electrode voltage supply unit 115. The counter electrode voltage supply unit 115 is formed in the substrate 101, and applies a predetermined voltage to the counter electrode 108 via the connection units 106 and the connection electrodes 103. In a case where a voltage to be applied to the counter electrode 108 is higher than a power supply voltage of the imaging element, the power supply voltage is boosted by a boosting circuit such as a charge pump to supply the predetermined voltage.

The readout circuits 116 are provided on the substrate 101 corresponding to each of the plurality of pixel electrodes 104, and read out signals corresponding to charges trapped by the corresponding pixel electrodes 104. The readout circuits 116 are configured, for example, of CCD and CMOS circuits, or a thin film transistor (TFT) circuit, and are shielded by the light shielding layer not shown in the drawing which is disposed in the insulating layer 102. The readout circuits 116 are electrically connected to the corresponding the pixel electrodes 104 via the connection units 105.

The buffer layer 109 is formed on the counter electrode 108 so as to cover the counter electrode 108. The sealing layer 110 is formed on the buffer layer 109 so as to cover the buffer layer 109. The color filters 111 are formed on the sealing layer 110 at positions corresponding to each of the pixel electrodes 104. The partition walls 112 are provided between the color filters 111, and are used for improving the light transmittance of the color filters 111.

The light shielding layer 113 is formed on the sealing layer 110 in a region other than the region where the color filters 111 and the partition walls 112 are provided, and prevents light from being incident to the photoelectric conversion film 107 formed outside an effective pixel region. The protective layer 114 is formed on the color filters 111, the partition walls 112, and the light shielding layer 113, and protects the entirety of the imaging element 100.

In the imaging element 100 configured as described above, light which has entered is incident on the photoelectric conversion film 107, and charges are generated in the photoelectric conversion film. The positive holes among the generated charges are trapped by the pixel electrodes 104, and voltage signals corresponding to the amount are output to the outside of the imaging element 100 using the readout circuits 116.

A method of producing the imaging element 100 is as follows. The connection units 105 and 106, the plurality of connection electrodes 103, the plurality of pixel electrodes 104, and the insulating layer 102 are formed on the circuit substrate in which the counter electrode voltage supply unit 115 and the readout circuits 116 are formed. The plurality of pixel electrodes 104 are disposed, for example, on the surface of the insulating layer 102 in a square lattice shape.

Next, the photoelectric conversion film 107 is formed on the plurality of pixel electrodes 104, for example, by the vacuum evaporation method. Next, the counter electrode 108 is formed on the photoelectric conversion film 107 under vacuum, for example, by the sputtering method. Next, the buffer layer 109 and the sealing layer 110 are sequentially formed on the counter electrode 108, for example, by the vacuum evaporation method. Next, after the color filters 111, the partition walls 112, and the light shielding layer 113 are formed, the protective layer 114 is formed, and the production of the imaging element 100 is completed.

### EXAMPLES

Examples will be shown below, but the present invention is not limited thereto.

(Synthesis of Compounds (D-1) and (D-2))

Compounds (D-1) and (D-2) were synthesized according to the method described in Inorganic Chemistry, 2003, 42, 6629-6647.

(Synthesis of Compound (D-3))

A compound (D-3) was synthesized according to the following scheme.

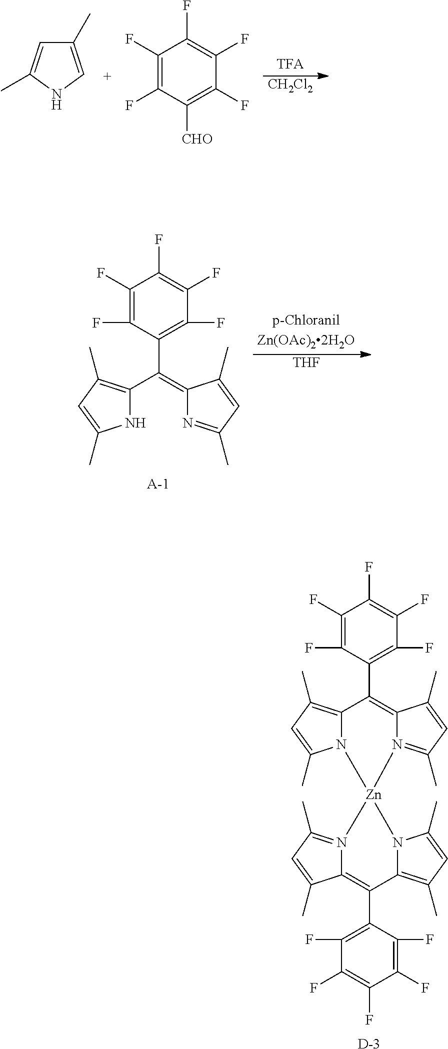

2,4-Dimethylpyrrole (5.10 g, 54.0 mmol) and pentafluorobenzaldehyde (5.00 g, 25.5 mmol) were added to methylene chloride (100 mL). Trifluoroacetic acid (TFA) (145 mg, 1.27 mmol) was added to the obtained mixed liquid and stirred, and the mixed liquid was reacted at room temperature for 1 hour. Triethylamine (0.5 mL) was added to the mixed liquid and concentrated, and the obtained product was purified by silica gel column (2% methanol/chloroform), whereby a compound (A-1) (7.15 g, yield 76%) was obtained.

The compound (A-1) (3.00 g, 8.19 mmol) was dissolved in tetrahydrofuran, and p-chloranil (2.01 g, 8.19 mmol) and zinc acetate (Zn (OAc)₂ 2H₂O) (4.49 g, 20.4 mmol) were added to the obtained solution. The obtained mixed liquid was stirred and reacted at room temperature for 1 hour. Then, the mixed liquid was concentrated, the obtained product was purified by silica gel column (2% methanol/chloroform), and the purified compound was recrystallized from methanol to obtain a compound (D-3) (1.64 g, yield 50%).

The obtained compound (D-3) was identified by mass spectrometry (MS).

MS(ESI⁺)m/z: 795.1 ([M+H]⁺)

(Synthesis of Compounds (D-4) to (D-11))

Compounds (D-4) to (D-11) were synthesized using the same reaction as described above.

A comparative compound (R-1) corresponding to a comparative compound was purchased from Luminescence Technology.

A comparative compound (R-2) was synthesized according to the method described in Organic Biomolecular Chemistry, 2010, 8, 4546-4553.

The structures of the obtained compounds (D-1) to (D-11) and the comparative compounds (R-1) to (R-2) are specifically shown below.

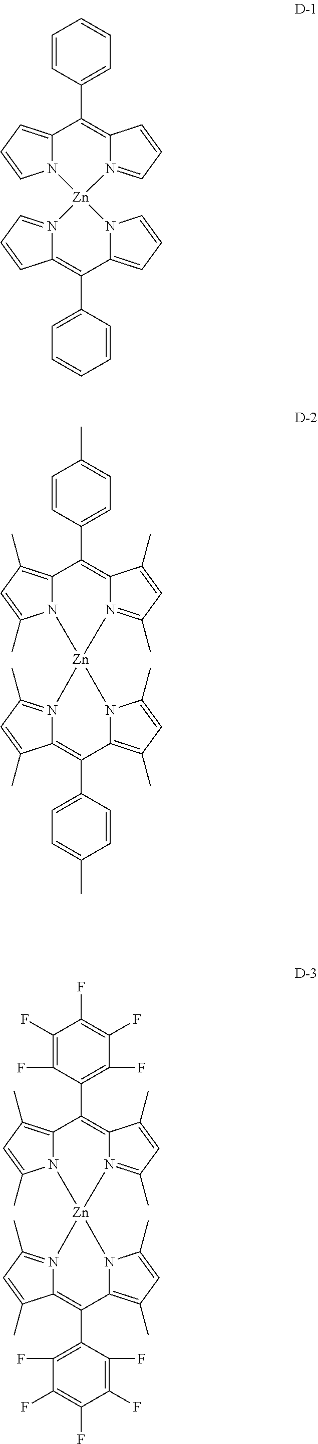

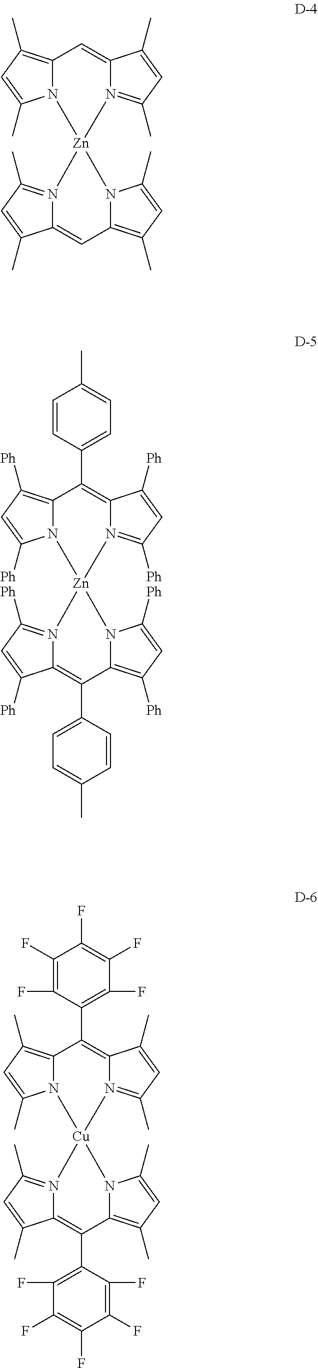

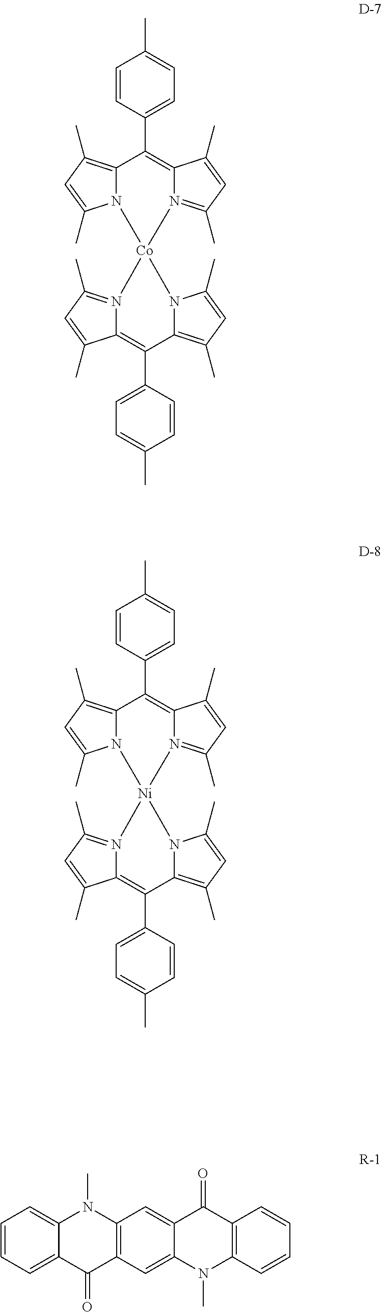

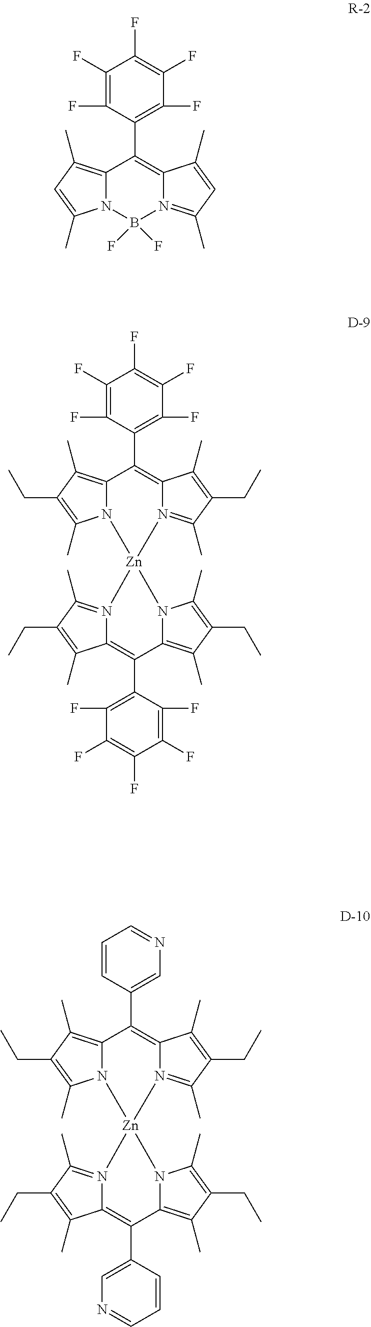

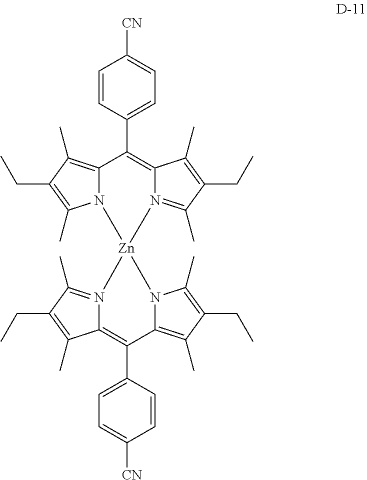

The n-type organic semiconductors used in Examples or Comparative Examples are shown below. The compound (NR-1) is C₆₀ (fullerene).

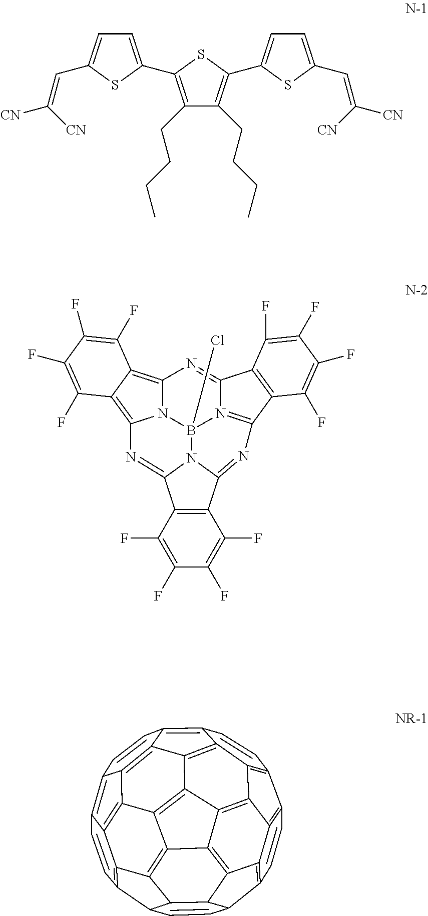

The maximum absorption wavelengths of the compounds used in Examples or Comparative Examples are shown in Table 1.

The maximum absorption wavelength is a value measured in a solution state (a solvent: chloroform) by adjusting the absorption spectrum of the compound to a concentration at which the light absorbance is 0.5 to 1.

TABLE 1

| | Maximum |
|---|---|
| | absorption |
| Compound | wavelength (nm) |
| D-1 | 483 |
| D-2 | 490 |
| D-3 | 501 |
| D-4 | 488 |
| D-5 | 558 |
| D-6 | 514 |
| D-7 | 498 |
| D-8 | 513 |
| D-9 | 523 |
| D-10 | 510 |
| D-11 | 513 |
| R-1 | 520 |
| R-2 | 519 |
| N-1 | 512 |
| N-2 | 554 |
| NR-1 | 443 |

<Production of Photoelectric Conversion Element>

The photoelectric conversion element of the form of FIG. 1A was produced using the obtained compound. That is, the photoelectric conversion element to be evaluated in the present example includes the lower electrode 11, the electron blocking film 16A, the photoelectric conversion film 12, and the upper electrode 15.

Also, a case of producing the photoelectric conversion film using the compound (D-1) as the p-type organic semiconductor and the compound (N-1) as the n-type organic semiconductor will be described in detail below.

Specifically, an amorphous ITO film was formed on the glass substrate by the sputtering method to form the lower electrode 11 (a thickness: 30 nm). Furthermore, a film of molybdenum oxide (MoOₓ) was formed on the lower electrode 11 by the vacuum evaporation method to form a molybdenum oxide layer (a thickness: 30 nm) as the electron blocking film 16A.

Furthermore, the compound (D-1) and the compound (N-1) were subjected to co-vapor deposition by the vacuum evaporation method so as to be respectively 50 nm in terms of single layer on a molybdenum oxide layer 16A to form a film in a state where the temperature of the substrate was controlled to 25 ° C., and the photoelectric conversion film 12 having the bulk hetero structure of 100 nm was formed. At this time, the formation speed of the photoelectric conversion film 12 was 1.0 Å/sec.

Furthermore, amorphous ITO film was formed on the photoelectric conversion film 12 by the sputtering method to form the upper electrode 15 (the transparent conductive film) (a thickness: 10 nm). After a SiO film was formed on the upper electrode 15 by vacuum evaporation method as the sealing layer, an aluminum oxide (Al₂O₃) layer was formed on the SiO film by an atomic layer chemical vapor deposition (ALCVD) method to produce the photoelectric conversion element. The element is referred to as an element (A).

The photoelectric conversion element (the element (A)) of each example shown in Table 2 below was produced according to the same procedure as described above except that the combination of the p-type organic semiconductor and the n-type organic semiconductor was changed as shown in Table 2.

<Evaluation>

(Evaluation of Responsiveness)

The following evaluation of responsiveness was performed using each obtained photoelectric conversion element (the element (A)).

Specifically, a voltage was applied to the photoelectric conversion element so that the photoelectric conversion efficiency to the maximum absorption wavelength of the photoelectric conversion film becomes 50%. Thereafter, a light emitting diode (LED) was instantaneously turned on to radiate light from the upper electrode (the transparent conductive film) side, and the photocurrent at that time was measured with an oscilloscope to obtain a rise time to signal intensities of 0% to 97%. The rise time of Comparative Example 1 (the element (A) produced by combining the compound (R-1) with the compound (N-2)) was set to 10, the relative value of the rise time of each element (A) was obtained.

Relative to Comparative Example 1, a case where the relative value of the rise time is less than 3 was set as “A”, a case of 3 or more and less than 5 was set as “B”, a case of 5 or more and less than 10 was set as “C”, and a case of 10 or more was set as “D”. For practical use, “A” or “B” is preferable, and “A” is more preferable.

The results are shown in Table 2 below.

(Evaluation of Dark Current Characteristic at Time of High-Speed Film Formation)

The photoelectric conversion elements (an element (B)) of examples shown in Table 2 below were produced in the same procedure as the element (A) except that the film formation speed of the photoelectric conversion film 12 was set as 3.0 Å/sec.

The dark current characteristics in a case of high-speed film formation were evaluated by using the obtained element (B). Specifically, a voltage was applied to the photoelectric conversion element so that the photoelectric conversion efficiency to the maximum absorption wavelength of the photoelectric conversion film becomes 50%, and in that state, the value of the dark current of the element (A) is set to 1. Also, regarding the element (B) made of a combination of the same p-type organic semiconductor and the n-type organic semiconductor, a value of the dark current of the element was measured in a state of applying a voltage such that the photoelectric conversion efficiency with respect to the maximum absorption wavelength of the photoelectric conversion film is 50%, and the relative value to the value of the dark current of the element (A) was obtained in the same manner.

A case where the relative value of the dark current of the element (B) to that of the element (A) is less than 1.5 was set as “A”, a case of 1.5 or more and less than 3 was set as “B”, a case of 3 or more and less than 5 was set as “C”, and a case of 5 or more was set as “D”. For practical use, “A” or “B” is preferable, and “A” is more preferable.

The results are shown in Table 2 below. In Table 2, the column “maximum absorption wavelength” represents the maximum absorption wavelength of the photoelectric conversion film.

Also, the column “relative value of light absorbance (light absorbance at maximum absorption wavelength is 1)” represents relative values of the light absorbance at a wavelength of 400 nm and at a wavelength of 650 nm in a case where the light absorbance at the maximum absorption wavelength of the photoelectric conversion film is 1.

The light absorbance of the photoelectric conversion film was measured by using a spectrophotometer UV-3600 manufactured by Shimadzu Corporation. Specifically, a film was produced on a 2.5 cm square glass substrate, the substrate was fixed to a film holder attached to the spectrophotometer, and the transmittance was measured to obtain the light absorbance.

In a case where the relative value is less than 0.01, the relative value was evaluated as 0.

TABLE 2

| | | | | Relative value of | | | |
|---|---|---|---|---|---|---|---|
| | | | | light absorbance | | | |
| | | | | (light absorbance | | | |
| | | | | at maximum | | | Dark current |
| | | | Maximum | absorption | | | characteristics |
| | Compound | | absorption | wavelength is | | | in case of |
| | represented by | n-Type organic | wavelength | set as 1) | | | high-speed film |
| | Formula (1) | semiconductor | (nm) | 400 nm | 650 nm | Responsiveness | formation |
| Example 1 | D-1 | N-2 | 497 | 0.05 | 0 | A | A |
| Example 2 | D-2 | N-2 | 518 | 0.06 | 0 | A | A |
| Example 3 | D-3 | N-2 | 520 | 0.09 | 0 | A | A |
| Example 4 | D-4 | N-2 | 502 | 0.05 | 0 | A | A |
| Example 5 | D-5 | N-2 | 570 | 0.09 | 0.02 | A | A |
| Example 6 | D-6 | N-2 | 520 | 0.12 | 0 | B | A |
| Example 7 | D-7 | N-2 | 504 | 0.08 | 0 | B | A |
| Example 8 | D-8 | N-2 | 533 | 0.18 | 0 | B | B |
| Example 9 | D-1 | N-1 | 492 | 0.15 | 0.04 | B | A |
| Example 10 | D-2 | N-1 | 505 | 0.17 | 0.04 | B | A |
| Example 11 | D-3 | N-1 | 518 | 0.19 | 0.03 | B | A |
| Example 12 | D-6 | N-1 | 516 | 0.25 | 0.07 | B | B |
| Example 13 | D-9 | N-2 | 540 | 0.05 | 0 | A | A |
| Example 14 | D-10 | N-2 | 521 | 0.07 | 0 | A | A |
| Example 15 | D-11 | N-2 | 526 | 0.08 | 0 | A | A |
| Example 16 | D-9 | N-1 | 531 | 0.05 | 0 | B | A |
| Comparative | R-1 | N-2 | 581 | 0.06 | 0.02 | D | C |
| Example 1 | | | | | | | |
| Comparative | R-2 | N-2 | 570 | 0.04 | 0 | D | D |
| Example 2 | | | | | | | |
| Comparative | R-1 | N-1 | 553 | 0.14 | 0.08 | C | D |
| Example 3 | | | | | | | |
| Comparative | D-1 | NR-1 | 581 | 0.37 | 0.11 | B | C |
| Example 4 | | | | | | | |

As shown in Table 2, it was confirmed that the photoelectric conversion element having the photoelectric conversion film including the compound represented by Formula (1), and the compound represented by Formula (2) or Formula (3) as the n-type organic semiconductor exhibits both excellent responsiveness and excellent dark current characteristic in a case of high-speed film formation.

Among these, it was confirmed, from a comparison between Examples 1 and 9 and Examples 6 and 12, that the photoelectric conversion element having the photoelectric conversion film including the compound represented by Formula (3) as the n-type organic semiconductor exhibits excellent responsiveness and excellent dark current characteristic in a case of high-speed film formation.

Among these, it was confirmed, from a comparison between Examples 1 to 8, that better responsiveness is exhibited in a case where M contains the compound represented by Formula (1) representing Zn.

On the other hand, in Comparative Examples 1 to 4 in which a combination of predetermined compounds was not used, a desired effect was not obtained.

<Production of Imaging Element>

The same imaging element as shown in FIG. 3 was produced. That is, 30 nm of an amorphous TiN film was formed on a CMOS substrate by a sputtering method, and was used as the lower electrode through patterning such that each pixel was present on the photodiode (PD) on the CMOS substrate through photolithography, and then the imaging element was produced similarly to the element (A) or the element (B) after the film formation of the electron blocking material. Evaluations of responsiveness of each of the obtained imaging elements and the dark current characteristic in a case of high-speed film formation were also carried out in the same manner, and the same results as those in Table 2 were obtained. As a result, it was found that the photoelectric conversion element of the present invention exhibits excellent performance also in the imaging element.

### EXPLANATION OF REFERENCES

10a, 10b: photoelectric conversion element

11: conductive film (lower electrode)

12: photoelectric conversion film

15: transparent conductive film (upper electrode)

16A: electron blocking film

16B: positive hole blocking film

100: pixel separation type imaging element

101: substrate

102: insulating layer

103: connection electrode

104: pixel electrode (lower electrode)

105: connection unit

106: connection unit

107: photoelectric conversion film

108: counter electrode (upper electrode)

109: buffer layer

110: sealing layer

111: color filter (CF)

112: partition wall

113: light shielding layer

114: protective layer

115: counter electrode voltage supply unit

116: readout circuit

200: photoelectric conversion element (hybrid type photoelectric conversion element)

201: inorganic photoelectric conversion film

202: n-type well

203: p-type well

204: n-type well

205: p-type silicon substrate

207: insulating layer

208: pixel electrode

209: organic photoelectric conversion film

210: common electrode

211: protective film

212: electron blocking film

What is claimed is:

1. A photoelectric conversion element comprising:
a conductive film;
a photoelectric conversion film; and
a transparent conductive film, in this order,
wherein the photoelectric conversion film contains a compound represented by Formula (1), and an n-type organic semiconductor, and
the n-type organic semiconductor contains at least one selected from the group consisting of a compound represented by Formula (2) and a compound represented by Formula (3), 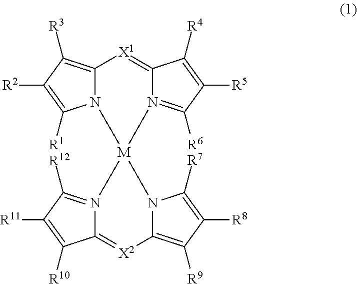
in Formula (1), R¹ to R¹² each independently represent a hydrogen atom or a substituent, X¹ and X² each independently represent a nitrogen atom or CR¹³, R¹³ represents a hydrogen atom or a substituent, and M represents a divalent metal atom, 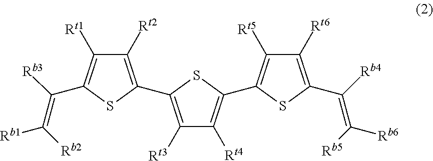
in Formula (2), R^{t1} to R^{t6} each independently represent a hydrogen atom or a substituent, R^{b1} to R^{b6} each independently represent a hydrogen atom or a substituent, and at least one of R^{b1}, . . . , or R^{b6} represents an electron attractive group, and 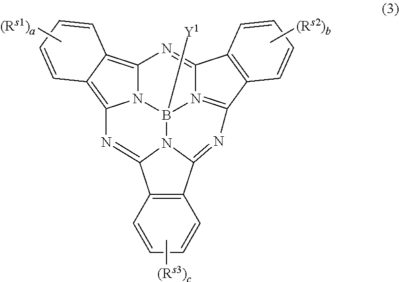
in Formula (3), R^{s1} to R^{s3} each independently represent a substituent, a to c each independently represent an integer of 0 to 4, and Y¹ represents a halogen atom, an alkoxy group, an aryloxy group, an alkylthio group, an arylthio group, a group having a carbonyloxy group, an amino group, an ethynyl group, or an ethenyl group.

2. The photoelectric conversion element according to claim 1,
wherein the n-type organic semiconductor contains the compound represented by Formula (3).

3. The photoelectric conversion element according to claim 1,
wherein M represents Zn, Cu, Co, Ni, Pt, Pd, Mg, or Ca.

4. The photoelectric conversion element according to claim 1,
wherein M represents Zn.

5. The photoelectric conversion element according to claim 1,
wherein a maximum absorption wavelength of the compound represented by Formula (1) is within a range of 480 to 600 nm.

6. The photoelectric conversion element according to claim 1,
wherein in a case where the photoelectric conversion film has a maximum absorption wavelength within a range of 480 to 600 nm and light absorbance of the photoelectric conversion film in the maximum absorption wavelength is 1, each of relative values of the light absorbance of the photoelectric conversion film at 400 nm and at 650 nm is 0.10 or less.

7. The photoelectric conversion element according to claim 1,
wherein a molecular weight of the compound represented by Formula (1) is 400 to 1200.

8. The photoelectric conversion element according to claim 1,
wherein the photoelectric conversion film has a bulk hetero structure.

9. The photoelectric conversion element according to claim 1, further comprising one or more interlayers between the conductive film and the transparent conductive film, in addition to the photoelectric conversion film.

10. An optical sensor comprising the photoelectric conversion element according to claim 1.

11. An imaging element comprising the photoelectric conversion element according to claim 1.

12. A compound represented by Formula (1-1), 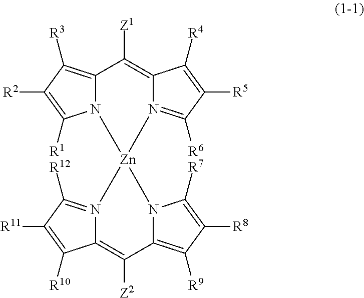
in Formula (1-1), R¹ to R¹² each independently represent a hydrogen atom, an alkyl group, an aryl group, or a heteroaryl group, and Z¹ and Z² each independently represent an aryl group which has a Hammett substituent constant σₚ exceeding 0 and may have a substituent, or a heteroaryl group which has a Hammett substituent constant σₚ exceeding 0 and may have a substituent.

13. The photoelectric conversion element according to claim 2,
wherein M represents Zn, Cu, Co, Ni, Pt, Pd, Mg, or Ca.

14. The photoelectric conversion element according to claim 2,
wherein M represents Zn.

15. The photoelectric conversion element according to claim 2,
wherein a maximum absorption wavelength of the compound represented by Formula (1) is within a range of 480 to 600 nm.

16. The photoelectric conversion element according to claim 2,
wherein in a case where the photoelectric conversion film has a maximum absorption wavelength within a range of 480 to 600 nm and light absorbance of the photoelectric conversion film in the maximum absorption wavelength is 1, each of relative values of the light absorbance of the photoelectric conversion film at 400 nm and at 650 nm is 0.10 or less.

17. The photoelectric conversion element according to claim 2,
wherein a molecular weight of the compound represented by Formula (1) is 400 to 1200.

18. The photoelectric conversion element according to claim 2,
wherein the photoelectric conversion film has a bulk hetero structure.

19. The photoelectric conversion element according to claim 2, further comprising one or more interlayers between the conductive film and the transparent conductive film, in addition to the photoelectric conversion film.

20. An optical sensor comprising the photoelectric conversion element according to claim 2.
